(19) United States
(12) Patent Application Publication
(10) Pub. No.: US 2021/0145452 A1
(43) Pub. Date: May 20, 2021

(54) HEMOSTATIC DEVICE

(71) Applicant: TERUMO KABUSHIKI KAISHA, Tokyo (JP)

(72) Inventors: Yuichi HIOKI, Fuji (JP); Ryo OKAMURA, Fujinomiya (JP); Fumi WATANABE, Hadano (JP); Satoshi WADA, Fujinomiya (JP); Masako MIYASHITA, Fujinomiya (JP)

(73) Assignee: TERUMO KABUSHIKI KAISHA, Tokyo (JP)

(21) Appl. No.: 17/160,875

(22) Filed: Jan. 28, 2021

Related U.S. Application Data
(63) Continuation of application No. PCT/JP2019/029819, filed on Jul. 30, 2019.

(30) Foreign Application Priority Data
Aug. 1, 2018 (JP) 2018-145365

(51) Int. Cl. A61B 17/135; A61B 17/132

(52) U.S. Cl. CPC A61B 17/135; A61B 2017/12004; A61B 17/1325

(57) ABSTRACT

A hemostatic device includes a covering member having a main body connected to an inflatable member, a first arm portion extending from the main body, and a second arm portion extending from the main body in a direction different from a longitudinal direction of extent of the first arm portion. The first arm portion is configured to be secured to the second arm portion by virtue of a part of the first arm portion passing between fingers of a patient while the second arm portion is wrapped around a limb of the patient. The second arm portion has a belt portion for wrapping around the patient's limb and a support portion connecting the main body and the belt portion and located on a straight line connecting the first arm portion and the inflatable member. The belt portion is securable to the support portion while wrapped around the patient's limb.

### CROSS-REFERENCES TO RELATED APPLICATIONS

This application is a continuation of International Patent Application No. PCT/JP2019/029819 filed on Jul. 30, 2019, which claims priority to Japanese Patent Application No. 2018-145365 filed on Aug. 1, 2018, the entire content of both of which is incorporated herein by reference.

### FIELD OF THE INVENTION

The present invention generally relates to a hemostatic device.

### BACKGROUND DISCUSSION

As a catheter procedure, a procedure is known in which a blood vessel (for example, radial artery) in an arm of a patient is punctured, and various elongated medical bodies or devices are introduced into the blood vessel via a puncture site formed in the blood vessel in the arm of the patient so as to perform treatment or therapy on a lesion area. An example of this is disclosed in Japanese Patent Application Publication No. 2008-119517. The catheter procedure utilizing the radial artery is referred to as transradial artery approach and is considered a useful technique for, for example, coronary artery access and lower limb artery access.

A radial artery located in an arm of a human body is connected to a palmar artery which bypasses a hand side. Therefore, currently, as a new method of transradial artery approach, a catheter procedure using distal transradial approach (dTRA) has been attempted to access the palmar artery (including a distal radial artery) from an anatomical snuffbox located on a dorsal side of the hand or from a position around the snuffbox, and perform treatment through the vascular access site.

### SUMMARY

Blood vessels such as palmar arteries located in the hand are located in places where there are many movable parts such as fingers. For this reason, a shape around the puncture site in the hand changes with movement of the hand. Therefore, when hemostasis is performed on the puncture site, a pressing member placed on the hand is preferably an inflatable member that follows the movement of the hand and can easily adjust a compressive force on the puncture site.

However, when the pressing member is the inflatable member, the inflatable member exerts a force to inflate from the inside to the outside of the inflatable member in an inflated state. For this reason, in a hemostatic device having the inflatable member, when a shape around a hemostatic site changes due to the movement of the hand, and a large gap is created between the hemostatic device and the puncture site, the inflatable member rises, and it may not be possible to properly maintain the compressive force of the inflatable member on the puncture site. Therefore, when hemostasis is performed on the puncture site of the hand, the hemostatic device having the inflatable member needs to suppress rising by the inflatable member acting in a direction away from the puncture site and to appropriately secure the inflatable member to the puncture site. In this way, it is considered that the hemostatic device having the inflatable member can appropriately maintain the compressive force of the inflatable member on the puncture site even when the shape around the puncture site is changed by the movement of the hand.

The hemostatic device disclosed here is capable of suppressing rising of an inflatable member from a site where bleeding is to be stopped on a hand and appropriately maintaining a compressive force of the inflatable member on the site where bleeding is to be stopped on the hand even when a patient moves the hand while the inflatable member is inflated.

A hemostatic device according to an aspect includes a covering member configured to cover a site on a hand of a patient where bleeding is to be stopped, a securing member connected to the covering member and configured to secure the covering member on the hand of the patient while the covering member is covering the site on the patient's hand where bleeding is to be stopped, and an inflatable member connected to the covering member and configured to be inflated by injection of a fluid. The covering member includes a main body to which the inflatable member is connected, a first arm portion extending away from the main body in a longitudinal direction of extent, and a second arm portion extending away from the main body in a direction different from the longitudinal direction of extent of the first arm portion. The first arm portion and the inflatable member are positioned so that a straight line connects the first arm portion and the inflatable member. The second arm portion includes a belt portion configured to be wrapped around a limb of the patient and a support portion that connects the main body and the belt portion, with the support portion being located on the straight line that connects the first arm portion and the inflatable member. The first arm portion is configured to be secured to the second arm portion by passing between fingers of the patient's hand in a state in which the second arm portion is wrapped around the limb of the patient. The belt portion of the second arm portion is configured to be secured to the support portion in the state in which the second arm portion is wrapped around the limb of the patient.

A hemostatic device according to another aspect includes a covering member configured to cover a site on a hand of a patient where bleeding is to be stopped, a securing member connected to the covering member and configured to secure the covering member on the hand of the patient while the covering member is covering the site on the patient's hand where bleeding is to be stopped, and an inflatable member connected to the covering member and configured to be inflated by injection of a fluid. The covering member includes a main body to which the inflatable member is connected, a first arm portion extending away from the main body in a longitudinal direction of extent, and a second arm portion extending away from the main body in a direction different from the longitudinal direction of extent of the first arm portion. The first arm portion is configured to be secured to the second arm portion by passing between fingers of the patient's hand in a state in which the second arm portion is wrapped around a limb of the patient. The inflatable member includes a first inflatable portion and a deformable auxiliary member, with the deformable auxiliary member and the deformable auxiliary member each having an outer shape, and the deformable auxiliary member having a smaller outer shape than the outer shape of the first inflatable portion. The first inflatable portion includes one portion located closer to the first arm portion and an other portion located farther from the first arm portion, and the auxiliary member overlaps the one portion of the first inflatable portion that is located closer to the first arm portion.

The hemostatic device can secure the main body, to which the inflatable member is connected, to the site where bleeding is to be stopped by wrapping the belt portion of the second arm portion around the limb of the patient. In addition, in the hemostatic device, when the first arm portion is secured to the second arm portion by passing between the fingers of the patient, it is possible to prevent the inflatable member from rising on the distal side of the hand while preventing position shift of the inflatable member. Further, when the hemostatic device is attached to the hand of the patient, it is possible to prevent the inflatable member from rising on the proximal side of the hand by connecting the belt portion of the second arm portion to the support portion located on the straight line connecting the first arm portion and the inflatable member. For this reason, in the hemostatic device, even when the hand is moved in a state in which the inflatable member is inflated, by securing the first arm portion and the second arm portion and securing the belt portion and the support portion, rising of the distal side (fingertip side) and the proximal side (wrist side) of the main body to which the inflatable member is connected is prevented. Thus, it is possible to appropriately maintain the compressive force of the inflatable member on the site where bleeding is to be stopped on the hand.

The hemostatic device according to the other aspect can secure the main body, to which the first inflatable portion is connected, to the site where bleeding is to be stopped by wrapping the second arm portion around the limb of the patient. Further, the hemostatic device can prevent the inflatable portion from rising on the distal side of the hand while preventing the first inflatable portion from being shifted by securing the first arm portion to the second arm portion through between the fingers of the patient. Further, when the first inflatable portion inflates, the hemostatic device inhibits the first inflatable portion from rising in a direction away from the site where bleeding is to be stopped on the hand of the patient by the auxiliary member. For this reason, even when the hand is moved in a state in which the inflatable member is inflated, the hemostatic device can prevent rising on the distal side (fingertip side) of the main body to which the inflatable member is connected and the distal side (fingertip side) of the first inflatable portion at the distal side of the hand where the shape of the hand changes most when the finger is moved by securing the first arm portion and the second arm portion and pressing the first inflatable portion using the auxiliary member. Thus, it is possible to appropriately maintain the compressive force of the first inflatable portion on the site where bleeding is to be stopped on the hand. In addition, in the hemostatic device, the deformable auxiliary member is disposed on the distal side of the hand (first arm side of the first inflatable portion) where the shape of the hand changes most when the finger is moved. Thus, when the shape of the auxiliary member changes according to the change in the shape of the hand, it is possible to appropriately maintain the compressive force of the first inflatable portion on the puncture site of the hand while preventing the main body from rising.

Another aspect involves a method of producing hemostasis at a puncture site on a hand of a patient where bleeding is to be stopped. The method involves positioning a main body of a covering member in overlying relation to the puncture site on the hand of the patient where bleeding is to be stopped so that an inflation member underlying the main body portion is positioned relative to the puncture site. The covering member also includes a first arm portion extending away from the main body in a longitudinal direction of extent, and a second arm portion extending away from the main body in a direction different from the longitudinal direction of extent of the first arm portion. The second arm portion includes a belt portion and a support portion that connects the belt portion to the main body, with the support portion being located on a straight line that connects the first arm portion and the inflatable member. The method additionally comprises: wrapping the band portion around a limb of the patient and securing the band portion to the support portion to maintain the second arm portion in a wrapped state on the limb of the patient; passing the first arm portion between two fingers of the hand of the patient while the second arm portion is in the wrapped state on the limb of the patient, and securing a part of the first arm portion which has passed between the two fingers of the hand of the patient to the second arm portion; and thereafter introducing a fluid into the inflatable member to inflate the inflatable member and apply a compressive force from the inflatable member to the puncture site to perform hemostasis.

### BRIEF DESCRIPTION OF THE DRAWINGS

FIG. 1 is a diagram illustrating a hemostatic device according to a first embodiment, and is a plan view seen from an outer surface side of a main body of a covering member.

FIG. 2 is an enlarged view illustrating a part of the covering member of the hemostatic device shown in FIG. 1.

FIG. 3 is a cross-sectional view of the hemostatic device taken along the section line 3-3 illustrated in FIG. 2, and illustrates an inflated state of an inflatable member.

FIG. 4 is a perspective view for description of a usage example of the hemostatic device according to the first embodiment.

FIG. 5 is a perspective view for description of a usage example of the hemostatic device according to the first embodiment.

FIG. 6 is a perspective view for description of a usage example of the hemostatic device according to the first embodiment.

FIG. 7 is a perspective view for description of a usage example of the hemostatic device according to the first embodiment.

FIG. 8 is a perspective view for description of a usage example of the hemostatic device according to the first embodiment.

FIG. 9 is a diagram schematically illustrating a part of a cross section taken along the section line 9-9 illustrated in FIG. 8.

FIG. 10 is a plan view of a hemostatic device according to a modification of the first embodiment.

FIG. 11 is a plan view of a hemostatic device according to a second embodiment.

FIG. 12 is a plan view of a hemostatic device according to a first modification of the second embodiment.

FIG. 13 is a perspective view for description of a usage example of the hemostatic device according to the first modification of the second embodiment.

FIG. 14 is a perspective view for description of a usage example of the hemostatic device according to the first modification of the second embodiment.

FIG. 15 is a plan view of a hemostatic device according to a second modification of the second embodiment.

FIG. 16 is a perspective view for description of a usage example of the hemostatic device according to the second modification of the second embodiment.

FIG. 17 is a perspective view for description of a usage example of the hemostatic device according to the second modification of the second embodiment.

### DETAILED DESCRIPTION

Set forth below with reference to the accompanying drawings is a detailed description of embodiments of a hemostatic device and method of use representing examples of the hemostatic device and method of use disclosed here. The dimensions or scales on the drawings may be exaggerated or different from actuality/reality for convenience of description and illustration. The following description does not limit the technical scope or the meaning of terms in the appended claims.

### First Embodiment

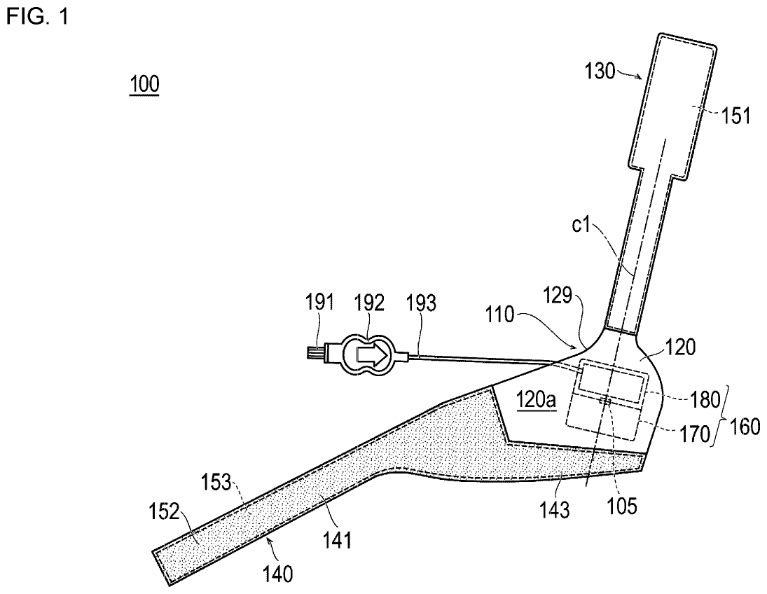

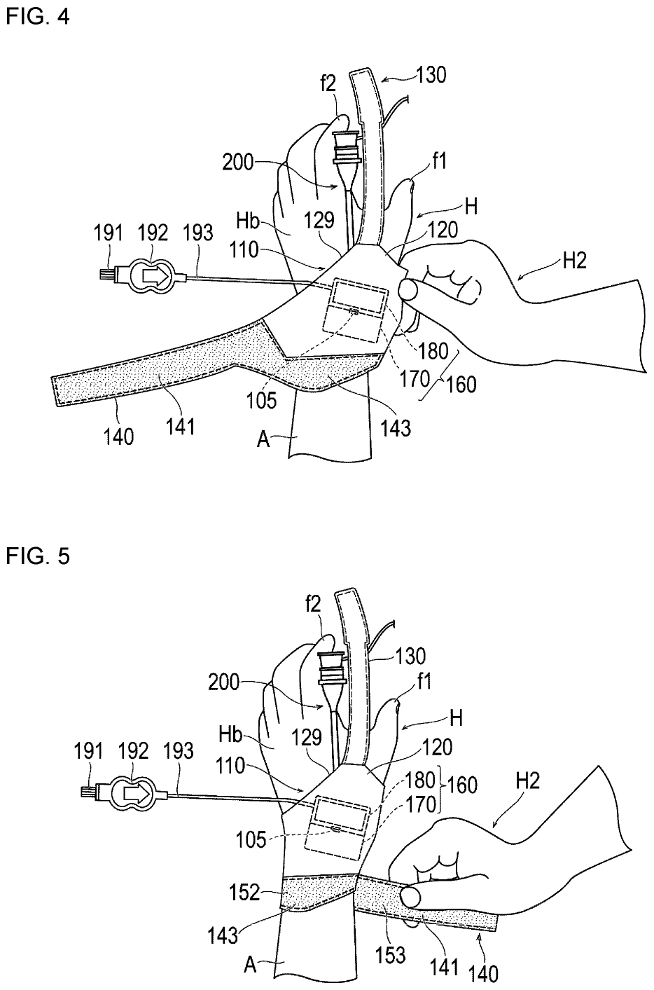

FIGS. 1 to 3 illustrate a hemostatic device 100 according to a first embodiment, and FIGS. 4 to 9 depict usage examples of the hemostatic device 100.

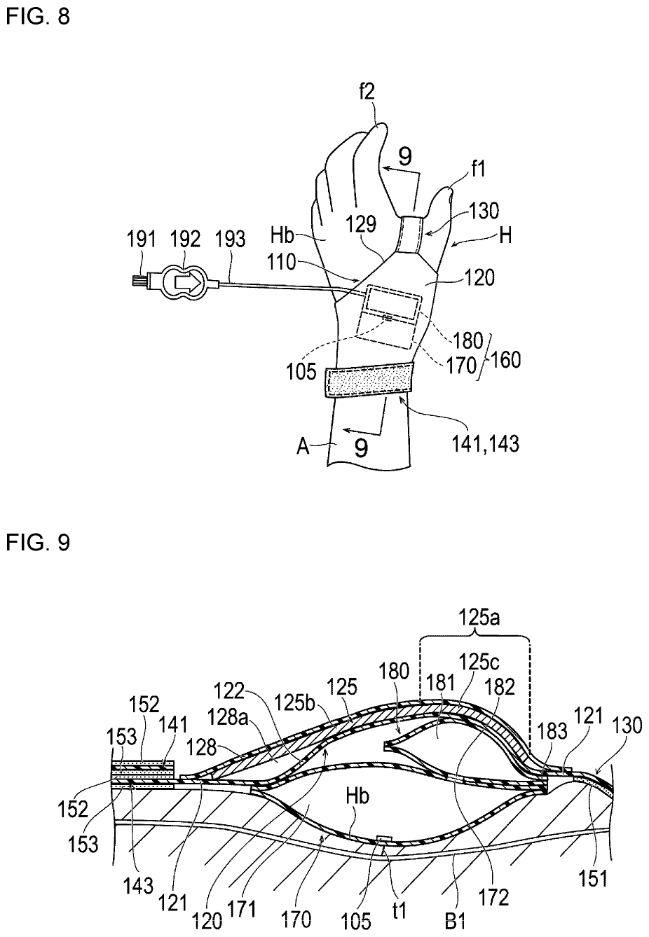

As illustrated by way of example in FIGS. 8 and 9, when removing a sheath tube of an introducer 200 (depicted in FIGS. 4-7) indwelling at a puncture site t1 (corresponding to a site where bleeding is to be stopped) formed on a radial artery side (for example, an artery around an anatomical snuffbox or a distal radial artery extending along a fingertip side of the snuffbox) of a palmar artery (deep palmar artery) B1 running on a side of a dorsal side Hb of a hand (for example, left hand or right hand) H located on the fingertip side of a forearm A of a patient, the hemostatic device 100 is used to perform hemostasis on the puncture site t1. The anatomical snuffbox is a cavity in the hand H located on the radial side of the forearm A when the patient spreads a thumb f1 of the hand H.

In brief, as illustrated in FIGS. 1, 2, and 9, the hemostatic device 100 includes a covering member (cover) 110 configured to be disposed to cover the puncture site t1 on the hand H of the patient, a plurality of securing members 151, 152, and 153 for securing the covering member 110 on the patient's hand while the covering member 110 covers the puncture site t1, and an inflatable member 160 connected to the covering member 110 and configured to be inflated by injection of a fluid.

In the description of the present specification, a distal side of the hand H of the patient refers to a side where the fingertip(s) of the hand H is disposed (upper side in FIG. 8).

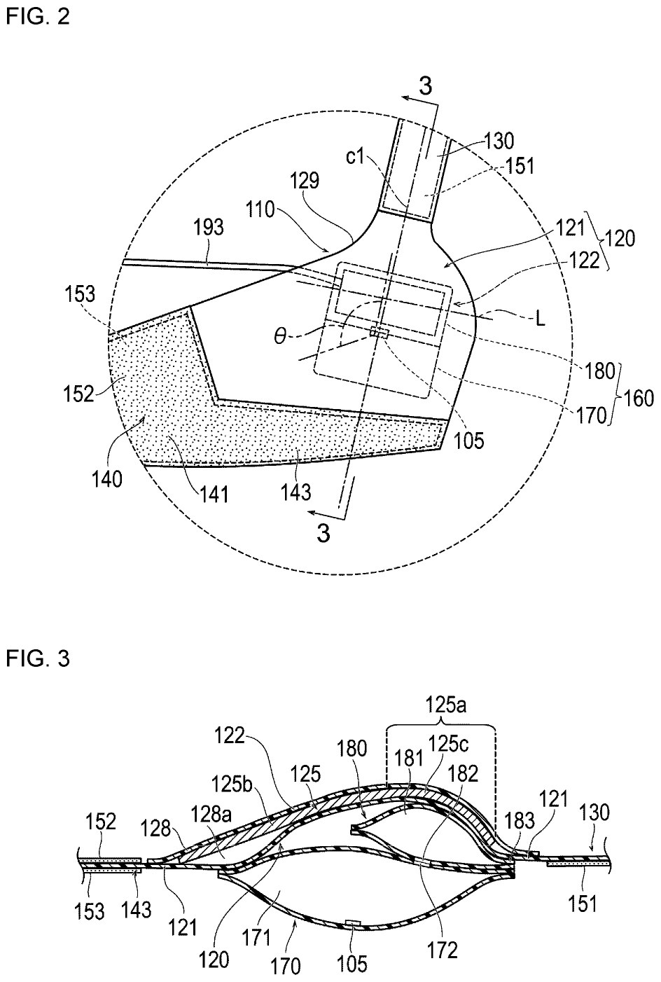

FIGS. 1 and 2 are plan views of the hemostatic device 100 as viewed from an outer surface 120a side of a main body 120 of the covering member 110. In each part of the hemostatic device 100, an “inner surface” is a surface on a side disposed to face a body surface of the patient when the hemostatic device 100 is attached to the patient, and an “outer surface” is a surface opposite to the inner surface (surface not disposed to face the body surface of the patient).

(Covering Member)

As illustrated in FIG. 1, the covering member 110 includes the main body 120 to which the inflatable member 160 is connected, a first arm portion (first extension portion) 130 having a first securing member (corresponding to the securing member) 151 and protruding from or extending away from the main body 120, and a second arm portion (second extension portion) 140 having a second securing member (corresponding to the securing member) 152 and protruding from or extending away from the main body 120 in a direction different from a longitudinal direction of the first arm portion 130.

As illustrated in FIGS. 1, 2, and 8, when the hemostatic device 100 is attached to the hand H of the patient to perform hemostasis, a part of the first arm portion 130 passes between the fingers f1 and f2 of the patient's hand while the second arm portion 140 is wrapped around a limb of the patient, and the first arm portion 130 is secured to the second arm portion 140.

As illustrated in FIG. 8, the limb around which the second arm portion 140 is wrapped is, for example, a portion including a part of the forearm A and a part of a wrist. In addition, the space between the fingers (inter-finger portion) through which a part of the first arm portion 130 passes is, for example, a space between the thumb f1 and the index finger f2 of the hand H of the patient. However, a position of the limb around which the second arm portion 140 is wrapped or a position of the inter-finger portion through which the first arm portion 130 is passed is not particularly limited in this way and others are possible.

As illustrated in FIG. 8, the first arm portion 130 is formed of a flexible band-shaped member that can extend from the side of the dorsal side Hb of the hand H to a palm Hp side of the hand H of the patient by passing between the thumb f1 and the index finger f2 when the hemostatic device 100 is attached to the hand H of the patient. A wide portion for facilitating holding of the first arm portion 130 by fingers is provided at an end portion of the first arm portion 130 disposed on the opposite side from the main body 120.

FIG. 1 shows that the second arm portion 140 includes a belt portion 141 for wrapping the second arm portion 140 around the limb of the patient, and a support portion 143 located on a straight line c1, which connects (extends between or intersects) the first arm portion 130 and the inflatable member 160, to connect the main body 120 of the covering member 110 and the belt portion 141 of the second arm portion 140. As shown in FIG. 1, the straight line c1 extends along the longitudinal extent of the first arm portion 130 (e.g., the center line of the first arm portion 130 in the illustrated embodiment), intersects the inflatable member 160 in the plan view of FIG. 1 and also intersects or passes through the support portion 143.

As illustrated in FIGS. 1 and 2, the straight line c1 can be defined as a straight line extending in parallel with an extending direction of the first arm portion 130 in a state where the first arm portion 130 and the second arm portion 140 are spread such as would be the case when the device 100 is lying on a flat horizontal surface.

As illustrated in FIG. 8, the belt portion 141 of the second arm portion 140 is secured to the support portion 143 with the belt portion 141 wrapped around the limb of the patient. The belt portion 141 and the support portion 143 are secured by a third securing member 153 disposed on the belt portion 141 and the second securing member 152 disposed on the support portion 143 (see FIG. 9).

As illustrated in FIGS. 5 and 7, the belt portion 141 is formed by a flexible band-shaped member that can be wrapped along a circumferential direction of the limb of the patient when the hemostatic device 100 is attached to the hand H of the patent. As illustrated in FIG. 5, when the hemostatic device 100 is attached to the hand H of the patent, the support portion 143 is disposed on a proximal side (wrist side when viewed from the hand H and lower side of FIG. 5) of the hand H of the patent with respect to the inflatable member 160. In the present embodiment, the belt portion 141 and the support portion 143 are integrally formed by one member. That is, as illustrated, the belt portion 141 and the support portion 143 are integrally formed as a single unitary piece. However, the belt portion 141 and the support portion 143 may be configured by connecting two or more members.

As illustrated in FIG. 2, the hemostatic device 100 can be configured such that an angle θ between the first arm portion 130 and the second arm portion 140 is 90° to 150° in a state where the arm portions 130 and 140 are extended substantially linearly before attaching the hemostatic device 100 to the patient. That is, the angle θ between the first arm portion 130 and the second arm portion 140 is 90° to 150° when the hemostatic device 100 is placed on a flat horizontal surface so that the hemostatic device 100 is as shown in the FIGS. 1 and 2 plan views. However, a particular direction in which each of the arm portions 130 and 140 protrudes from the main body 120 is not particularly limited as long as the inflatable member 160 can be disposed at the puncture site t1 in a state where the first arm portion 130 is disposed between predetermined fingers and the second arm portion 140 is wrapped around the limb.

A proximal end of the first arm portion 130 (end portion of the first arm portion 130 on the main body 120 side) can be disposed, for example, at a position 200 mm to 250 mm away from a marker portion 105 of the main body 120. In addition, a width of the first arm portion 130 can be, for example, 5 mm to 20 mm. In addition, a proximal end (proximal-most end) of the second arm portion 140 (end portion of the second arm portion 140 on the main body 120 side) can be disposed, for example, at a position 250 mm to 350 mm away from the marker portion 105 of the main body 120.

As illustrated in FIGS. 2 and 3, the main body 120 of the covering member 110 has a first region 121 and a second region 122. The second region 122 of the main body 120 is formed or located in a part of the main body 120 different from the first region 121. The inflatable member 160 is disposed on the second region 122 of the main body 120.

As illustrated in FIG. 3, the second region 122 of the main body 120 has a support member 125 having a higher rigidity than that of the first region 121 of the main body 120. Here, as illustrated in FIG. 2, the first region 121 of the main body 120 surrounds the support member 125 having a higher rigidity than that of the first region 121. That is, the support member 125 is surrounded by the first region 121 that is more flexible than the support member 125. Therefore, when the inflatable member 160 (a first inflatable portion 170 and a second inflatable portion 180) located on the inner surface side of the support member 125 inflates in the second region 122, an inflating force from the inside to the outside of the inflatable member 160 is hardly transmitted from the support member 125 to the first region 121, and thus it is possible to suppress formation of a gap between a surface of the dorsal side Hb of the hand H of the patient and the main body 120 at a peripheral edge of the main body 120.

As illustrated in FIG. 2, the first region 121 of the main body 120 surrounds the periphery of the second region 122 of the main body 120. As illustrated in FIG. 3, the support member 125 is inserted into or positioned in an insertion portion 128a disposed on an outer surface side of the second region 122 (surface opposite to a side where each of the inflatable portions 170 and 180 is disposed in the second region 122). The outer surface side of the second region 122 is a surface on an upper side of FIG. 3.

The insertion portion 128a is a space formed between the cover member 128 disposed to cover a part of the outer surface of the main body 120 and the main body 120. The insertion portion 128a is specifically illustrated and identified only in FIGS. 3 and 9.

An opening is formed between the main body 120 and the insertion portion 128a on a side portion side intersecting the straight line c1 on the plan view illustrated in FIG. 2. The insertion portion 128a communicates with or opens to the outside through the opening. The support member 125 can be inserted into the insertion portion 128a through the opening. The insertion portion 128a is thus a pocket that receives the support member 125.

The cover member 128 can be connected to the covering member 110, for example, by adhesion or welding.

As illustrated in FIG. 3, the support member 125 has a curved portion 125a formed on the first arm portion 130 side of the support member 125. The curved portion 125a has a cross-sectional shape that protrudes or curves upward so as to be separated from the main body 120 on the cross-sectional view illustrated in FIG. 3. The first arm portion 130 side refers to a side of the support member 125 close to the first arm portion 130 with reference to a center position (position where the marker portion 105 is disposed in the present embodiment) on the plan view of the inflatable member 160 illustrated in FIG. 2. The curved portion 125a possesses a concave surface that faces toward the inflatable member 160 and an oppositely disposed convex surface that faces away from the inflatable member 160 as shown in FIG. 3.

The support member 125 has an inclined portion 125b extending from the curved portion 125a toward the support portion 143 side. The inclined portion 125b extends substantially linearly toward the support portion 143 side.

The support member 125 may be disposed, for example, on the inner surface side of the main body 120 of the covering member 110 (surface on which each of the inflatable portions 170 and 180 is disposed, which is a surface on a lower side of FIG. 3). When the support member 125 is disposed in this way, each of the inflatable portions 170 and 180 can be secured to the support member 125, for example. Further, connection of the cover member 128 to the main body 120 can be omitted.

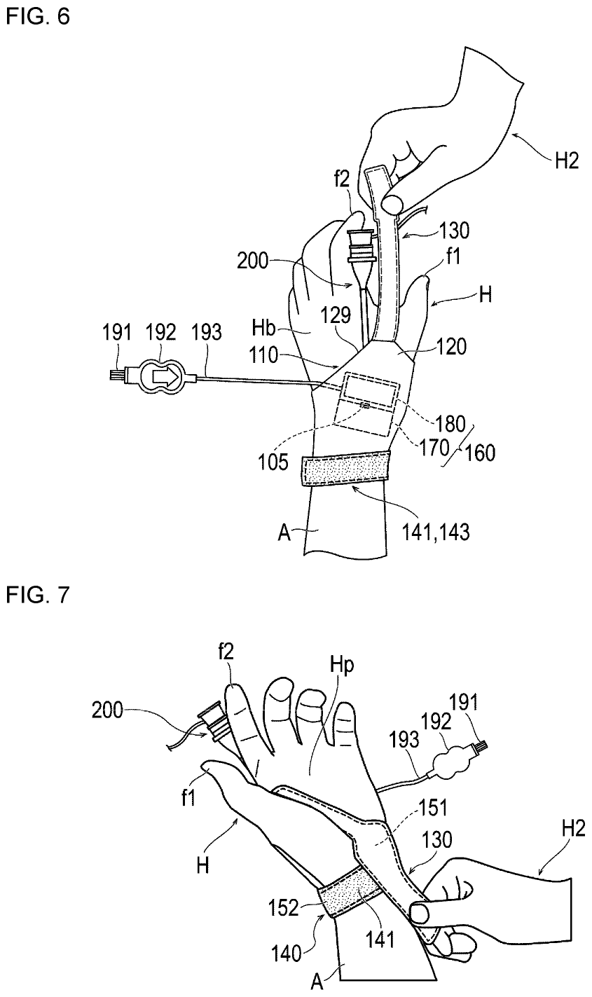

In addition, the peripheral edge of the main body 120 includes a sheath indwelling portion 129 having a shape recessed in a plan view toward the curved portion 125a side of the support member 125 at a position on the distal side (fingertip side) of an extension of a straight line L1 (see FIG. 2) passing through an apex 125c (a portion where the curved portion 125a is most distant from an inner surface of the inflatable member 160 in a state before the inflatable member 160 is inflated) of the curved portion 125a of the support member 125 between the first arm portion 130 and the second arm portion 140. The straight line L1 passes through the apex 125c and extends in the width-wise direction (perpendicular to the longitudinal extent of the support member 125). In addition, the curved portion 125a of the support member 125 forms a convex portion (convex surface) protruding in a direction away from the inflatable member 160 on the main body 120 of the covering member 110 in the state before the inflatable member 160 is inflated. For this reason, when the inflatable member 160 is inflated, the sheath indwelling portion 129 is recessed toward the curved portion 125a side of the support member 125 forming the convex portion on the main body 120, so that a distance between the peripheral edge of the main body 120 and the curved portion 125a is shortened. Thus, a gap portion is likely to be formed between the surface of the dorsal side Hb of the hand H of the patient and the covering member 110. Therefore, an operator attaches the hemostatic device 100 such that the sheath tube of the introducer 200 (see FIGS. 5 and 6) is located at the sheath indwelling portion 129 of the main body 120, so that the sheath tube can be easily removed even after the inflatable member 160 of the hemostatic device 100 is inflated. When the inflatable member 160 is inflated, the curved portion 125a side of the support member 125 wraps the end portion of the inflatable member 160 on the first arm portion 130 side, so that the compressive force of the inflatable member 160 is directed toward the center side of the support member 125. Thus, the compressive force of the inflatable member 160 is inhibited from escaping to the outside of the support member 125. For this reason, in the sheath indwelling portion 129, even when the inflatable member 160 inflates, and the gap portion is formed around the sheath indwelling portion 129 between the surface of the dorsal side Hb of the hand H of the patient and the covering member 110 so that the sheath tube can be easily removed, a decrease in the compressive force on the puncture site t1 by the inflatable member 160 is suppressed. Thus, it is possible to maintain the compressive force of the inflatable member 160 suitable for the puncture site t1.

The first region 121 and the second region 122 of the main body 120 are integrally formed by one member. That is, as illustrated, the first and second regions 121, 122 of the main body 120 are integrally formed as a single unitary piece. However, the first region 121 and the second region 122 may be configured by connecting different members.

In the second region 122 of the main body 120, the cover member 128, the support member 125, the first inflatable portion 170, and the second inflatable portion 180, a portion overlapping the marker portion 105 in the plan view illustrated in FIG. 2 and surroundings thereof are preferably transparent (including colored transparent, colorless transparent, and translucent). By adopting such a configuration, the operator can visually recognize the puncture site t1 from the outer surface side of the main body 120 even when the marker portion 105 is superposed on the puncture site t1.

In the present embodiment, the covering member 110 includes the main body 120, the first arm portion 130, and the second arm portion 140 (the belt portion 141 and the support portion 143), each of which is a separate member. When respective portions of the covering member 110 are configured as separate members in this way, each of the main body 120, the first arm portion 130, and the second arm portion 140 can be connected by, for example, adhesion, welding, etc.

The main body 120 is preferably made of a material different from that of the first arm portion 130 and the second arm portion 140. In this way, the main body 120 is easily deformed due to a change in physical property at each boundary between the first arm portion 130 and the second arm portion 140, and thus the first arm portion 130 and the second arm portion 140 can be easily disposed on the patient in a state where the main body 120 is disposed at the puncture site t1 located on the hand H of the patient. Further, a material from which the main body 120 is fabricated is preferably more rigid than materials forming the first arm portion 130 and the second arm portion 140. In this way, when the inflatable member 160 is inflated, the main body 120 can suppress rising of the main body 120 by the inflatable member 160 due to the rigidity of the main body 120. In addition, the first arm portion 130 and the second arm portion 140 are configured to be more flexible than the main body 120, so that when the hemostatic device 100 is attached to the patient, the covering member 110 can be easily attached along the shape of the hand H of the patient (i.e., the covering member 110 can easily follow the shape or contour of the patient's hand H).

Further, in the present embodiment, in the covering member 110, the main body 120, the first arm portion 130, and the second arm portion 140 (the belt portion 141 and the support portion 143) are configured as different members, respectively. However, in the covering member 110, any part of the main body 120, the first arm portion 130, and the second arm portion 140 may be integrally formed of one member.

The material used for the main body 120 of the covering member 110 is not particularly limited. Examples of such materials include polyvinyl chloride, polyolefin such as polyethylene, polypropylene, polybutadiene, or ethylene-vinyl acetate copolymer (EVA), polyester such as polyethylene terephthalate (PET) or polybutylene terephthalate (PBT), polyvinylidene chloride, silicone, polyurethane, various thermoplastic elastomers such polyamide elastomer, polyurethane elastomer, and polyester elastomer, nylon, nylon elastomer, or any combination thereof (blended resin, polymer alloy, laminate, etc.).

The material used for the cover member 128 is not particularly limited, and examples include the same materials as those exemplified as the material of the main body 120 of the covering member 110.

It is preferable that the material used for the support member 125 has a higher rigidity than that of the material used for the first region 121 of the main body 120 of the covering member 110. Examples of such a material include acrylic resin, polyvinyl chloride (especially hard polyvinyl chloride), polyolefin such as polyethylene, polypropylene, or polybutadiene, polystyrene, poly-(4-methylpentene-1), polycarbonate, ABS resin, polymethylmethacrylate (PMMA), polyacetal, polyacrylate, polyacrylonitrile, polyvinylidene fluoride, ionomer, acrylonitrile-butadiene-styrene copolymer, polyester such as polyethylene terephthalate (PET) or polybutylene terephthalate (PBT), butadiene-styrene copolymer, aromatic or aliphatic polyamide, fluorine-based resin such as polytetrafluoroethylene, etc.

the material used for the first arm portion 130 and the second arm portion 140 of the covering member 110 is not particularly limited. Examples thereof may include the same materials as the materials exemplified as the main body 120 of the covering member 110, woven fabric, nonwoven fabric, felt, cloth, knitted fabric, and paper.

(Securing Member)

as illustrated in FIGS. 1, 2, and 3, the first securing member 151 is disposed on the inner surface of the first arm portion 130. The first securing member 151 is formed on a female side of a surface fastener.

the second securing member 152 is disposed on an outer surface of the second arm portion 140 (an outer surface of the belt portion 141 and an outer surface of the support portion 143). The second securing member 152 is formed on a male side of the surface fastener.

the third securing member 153 is disposed on an inner surface of the second arm portion 140 (an inner surface of the belt portion 141 and an inner surface of the support portion 143). The third securing member 153 is formed on the female side of the surface fastener.

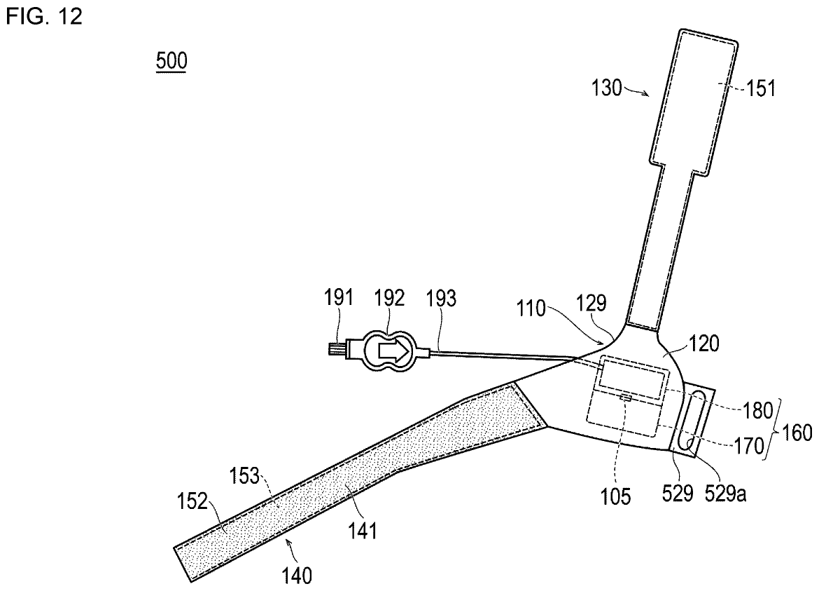

A specific configuration of each of the securing members 151, 152, and 153 is not limited as long as the second arm portion 140 wrapped around the limb of the patient can be secured to the support portion 143, and the first arm portion 130 passed between the thumb f1 and the index finger f2 can be secured to the second arm portion 140. For example, when each of the securing members 151, 152, and 153 includes the surface fastener, the male side and the female side of the surface fastener may be interchanged. The surface fastener is a fastener that is removable in terms of surface, and is, for example, MAGIC TAPE (registered trademark) or WELCRO (registered trademark). In addition, each of the securing members 151, 152, and 153 may be, for example, a snap, a button, a clip, a member 529 having a hole 529a as described in the embodiments described later (see FIG. 12), etc.

(Inflatable Member)

As illustrated in FIGS. 2 and 3, the inflatable member 160 includes the first inflatable portion 170 and a deformable auxiliary member 180 having a smaller outer shape (outer shape as seen in plan view like FIG. 2) than that of the first inflatable portion 170.

The auxiliary member 180 overlaps the first inflatable portion 170 on the first arm portion 130 side of the first inflatable portion 170. The first arm portion 130 side refers to a side of the first inflatable portion 170 that is close to the first arm portion 130 with reference to a center position (position where the marker portion 105 is disposed in the present embodiment) on the plan view of the inflatable member 160 illustrated in FIG. 2.

The first inflatable portion 170 has a lumen 171 into which a fluid can be injected, and a communication hole 172 formed at a position facing the auxiliary member 180.

The auxiliary member 180 is a second inflatable portion that is inflated by injection of a fluid. Hereinafter, the auxiliary member 180 is referred to as a second inflatable portion.

The second inflatable portion 180 has a lumen 181 into which a fluid can be injected, and a communication hole 182 formed at a position facing the first inflatable portion 170.

The lumen 181 of the second inflatable portion 180 communicates with the lumen 171 of the first inflatable portion 170 through the communication hole 182 of the second inflatable portion 180 and the communication hole 172 of the first inflatable portion 170.

The inflatable member 160 is fixed to the covering member 110 on the curved portion 125a side of the support member 125. As illustrated in FIG. 3, the second inflatable portion 180 is fixed to the inner surface of the covering member 110 (the inner surface of the main body 120 of the covering member 110). An end portion 183 located on the first arm portion 130 side of the second inflatable portion 180 is fixed to the inner surface of the covering member 110. In addition, the first inflatable portion 170 is connected to the second inflatable portion 180 near a central portion excluding a peripheral edge of the second inflatable portion 180. In the first inflatable portion 170, only a certain range around each communication hole 172, 182 located near the central portion of the second inflatable portion 180 is fixed to the second inflatable portion 180. For this reason, the first inflatable portion 170 is not directly connected to the covering member 110, and is indirectly connected to the covering member 110 via the second inflatable portion 180.

The inflatable member 160 may be fixed to the inner surface of the covering member 110 at a position different from that shown in FIG. 3 as long as the inflatable member 160 is fixed to the covering member 110 and is positioned on the curved portion 125a side of the support member 125. Specifically, a part or the whole of the outer surface side of the second inflatable portion 180 may be fixed to the inner surface of the covering member 110 on the curved portion 125a side of the support member 125. Even in such a configuration, in the hemostatic device 100, the compressive force on the first arm portion 130 side of the inflatable member 160 becomes stronger due to securing of the support member 125 and the covering member 110. Thus, even when a gap is generated between the first arm portion 130 and the surface of the hand H due to movement of a finger of the patient or bending of the wrist, it is possible to prevent a decrease in compressive force of the inflatable member 160.

The first inflatable portion 170 has a substantially square shape on the plan view illustrated in FIG. 2. The second inflatable portion 180 has a substantially rectangular shape that includes a set of long sides having substantially the same length as that of one side of the first inflatable portion 170 and a set of short sides having a length which is approximately half a length of one side of the first inflatable portion 170.

The lumen 181 of the second inflatable portion 180 communicates with a lumen of a tube 193 for supplying a fluid such as air to the second inflatable portion 180. As illustrated in FIG. 2, the tube 193 is connected to the second inflatable portion 180 on the second arm portion 140 side of the second inflatable portion 180. The tube 193 is pulled out to the outside of the main body 120 through the inner surface side of the main body 120 of the covering member 110. A position where the tube 193 is pulled out from the second inflatable portion 180 is not particularly limited. However, as illustrated in FIG. 2, when the tube 193 is pulled out to the second arm portion 140 side, so that the hemostatic device 100 is attached to the patient, the tube 193 is disposed laterally to the hand H (in a direction intersecting a direction in which the finger of the hand H extends) (see FIG. 5). For this reason, when the hemostatic device 100 is attached to the patient, the tube 193 can be prevented from interfering with the introducer 200.

As illustrated in FIGS. 2 and 3, the hemostatic device 100 has the marker portion 105 for aligning the inflatable member 160 with respect to the puncture site t1.

The marker portion 105 is disposed at a position corresponding to a substantially center position (center position on the plan view illustrated in FIG. 2) of the first inflatable portion 170.

As illustrated in FIG. 3, for example, the marker portion 105 can be disposed on an inner surface of a side surface (inner surface) of the first inflatable portion 170 disposed to face the body surface. However, the marker portion 105 may be disposed, for example, on an internal surface of a surface (outer surface) opposite to the side surface of the first inflatable portion 170 disposed to face the body surface or an external surface thereof, an internal surface or an external surface of the main body 120 of the covering member 110, an internal surface or an external surface of the support member 125, an internal surface or an external surface of the cover member 128, etc. In addition, when a center portion of the first inflatable portion 170 and an end portion of the second inflatable portion 180 (end portion on the support portion 143 side) are disposed to overlap each other on the cross-sectional view illustrated in FIG. 3, the marker portion 105 may be disposed on an external surface of the end portion of the second inflatable portion 180.

The marker portion 105 preferably includes, for example, a transparent central portion and a colored linear frame portion surrounding the central portion. In this way, the operator can dispose or position the marker portion 105 at the puncture site t1 while confirming the puncture site t1 through the transparent central portion of the marker portion 105. For this reason, the operator can easily dispose the center position of the first inflatable portion 170 at the puncture site t1 using the marker portion 105. The marker portion 105 may be, for example, formed only by the colored central portion without having the frame portion. Further, a specific shape and color of the marker portion 105, a formation method on each portion of the hemostatic device 100, etc. are not particularly limited.

In the present embodiment, the first inflatable portion 170 is formed of two sheet-shaped members. For example, the first inflatable portion 170 can be formed by forming the lumen 171 between two sheet-shaped members each possessing a substantially rectangular shape and bonding outer peripheral edges of the two sheet-shaped members in this state. Similarly to the first inflatable portion 170, the second inflatable portion 180 can be formed of two substantially rectangular sheet-shaped members bonded together.

A method of bonding the sheet-shaped members forming the first inflatable portion 170, and a method of bonding the sheet-shaped members forming the second inflatable portion 180 are not particularly limited. For example, it is possible to adopt adhesion or welding. Further, a method of connecting the second inflatable portion 180 and the main body 120 of the covering member 110 is not particularly limited. For example, adhesion or welding can be adopted. Further, a method of securing the periphery of the communication hole 172 of the first inflatable portion 170 and the periphery of the communication hole 182 of the second inflatable portion 180 is not particularly limited. For example, adhesion or welding can be adopted.

The first inflatable portion 170 and the second inflatable portion 180 may not have a structure in which a plurality of sheet-shaped members is bonded. The first inflatable portion 170 and the second inflatable portion 180 may be formed of, for example, one bag-shaped member in which a space (interior) into which a fluid can flow is formed.

A material used for the first inflatable portion 170 and the second inflatable portion 180 is not particularly limited, and examples may include the same materials as those discussed above as the material for fabricating the main body 120 of the covering member 110.

(Injection Portion)

As illustrated in FIG. 1, the hemostatic device 100 has an injection portion 191 for injecting a fluid into the inflatable member 160 (the first inflatable portion 170 and the second inflatable portion 180).

The injection portion 191 includes a connector having an incorporated check valve. A syringe can be connected to the injection portion 191.

A cushioning member 192 having an inflatable space is disposed between the injection portion 191 and the inflatable member 160. The cushioning member 192 includes a flexible bag-shaped member having a space formed inside. As illustrated in FIG. 1, the cushioning member 192 may be provided with an arrow-shaped marker indicating a direction in which the syringe is inserted into the injection portion 191.

The injection portion 191 is connected to one end side of the cushioning member 192. A lumen of the injection portion 191 communicates with a space in the cushioning member 192. However, while the check valve incorporated in the injection portion 191 is closed, communication between the lumen of the injection portion 191 and the space in the cushioning member 192 is cut off.

A flexible tube 193 is connected to the other end side of the cushioning member 192. That is, the flexible tube 193 extends from the cushioning member 192 to the inflatable member 160. A lumen of the tube 193 communicates with the space in the cushioning member 192. Further, in the tube 193, the other end portion opposite to one end portion connected to the cushioning member 192 is connected to the second inflatable portion 180. The lumen of the tube 193 communicates with the lumen 181 of the second inflatable portion 180.

The other end portion of the tube 193 can be connected to the second inflatable portion 180 using, for example, an adhesive, etc. while being interposed between the two sheet-shaped members forming the second inflatable portion 180. In the sheet-shaped members forming the second inflatable portion 180, for example, convex portions partially protruding outward from the sheet-shaped members may be formed at parts at which the tube 193 is interposed therebetween.

To inflate the first inflatable portion 170 and the second inflatable portion 180, the operator inserts a distal tubular portion of a syringe into the injection portion 191 to open the check valve. The operator injects air in the syringe into the lumen 181 of the second inflatable portion 180 by pushing a plunger of the syringe with the check valve of the injection portion 191 open.

When air is injected into the lumen 181 of the second inflatable portion 180, the second inflatable portion 180 inflates. Further, the air injected into the lumen 181 of the second inflatable portion 180 flows into the lumen 171 of the first inflatable portion 170 via the communication hole 182 of the second inflatable portion 180 and the communication hole 172 of the first inflatable portion 170. When air flows into the lumen 171 of the first inflatable portion 170, the first inflatable portion 170 inflates. When the first inflatable portion 170 and the second inflatable portion 180 inflate, the cushioning member 192 communicating with the lumen 181 of the second inflatable portion 180 via the tube 193 inflates.

The space in cushioning member 192 and the lumen 181 of the second inflatable portion 180 are in communication with each other via the tube 193 at all times. The injection portion 191 maintains the check valve incorporated in the injection portion 191 in a closed state when the syringe is not inserted into the injection portion 191 to prevent air from leaking from the injection portion 191. For this reason, when the internal pressure of the inflatable member 160 increases due to movement of the hand H of the patient, etc., the air in the lumen 171 of the first inflatable portion 170 and the lumen 181 of the second inflatable portion 180 moves to the cushioning member 192 side which is not pressed against the puncture site t1 by the covering member 110. In this way, the compressive force applied to the puncture site t1 by the inflatable member 160 is adjusted, and thus the compressive force to the puncture site t1 by the inflatable member 160 can be appropriately maintained.

To contract or deflate the first inflatable portion 170 and the second inflatable portion 180, the operator inserts the distal tubular portion of the syringe into the injection portion 191 and pulls the plunger of the syringe. By performing the above operation, the operator can discharge the air in the first inflatable portion 170 and the air in the second inflatable portion 180 to the syringe.

When the operator, etc. inflates the inflatable member 160, the operator, etc. can visually confirm that the first inflatable portion 170 and the second inflatable portion 180 can be pressurized without leakage of air by confirming expansion of the cushioning member 192.

Next, a usage example of the hemostatic device 100 will be described with reference to FIGS. 4 to 9.

FIG. 4 illustrates a state after performing various procedures by inserting the sheath tube of the introducer 200 into the distal radial artery side of the palmar artery B1 via the puncture site t1 (see FIG. 9) formed on the dorsal side Hb of the hand (left hand) H of the patient. In addition, FIG. 4 illustrates a state in which a part of the sheath tube of the introducer 200 is pulled out from the puncture site t1 after completing the above procedure(s).

At the start of hemostasis, as illustrated in FIG. 4, the operator, etc. disposes the main body 120 of the covering member 110 so as to overlap the side on the dorsal side Hb of the hand H. In this instance, the marker portion 105 formed at a substantially center position of the first inflatable portion 170 is disposed at the puncture site t1. The hand H2 illustrated in FIGS. 4 to 7 indicates a hand of the operator, etc. who attaches the hemostatic device 100 to the patient.

Subsequently, as illustrated in FIG. 5, the operator, etc. wraps the belt portion 141 of the second arm portion 140 along the limb of the patient to overlap the belt portion 141 on the support portion 143. The operator, etc. brings the third securing member (female side of the surface fastener) 153 disposed on the inner surface side of the belt portion 141 into contact with the second securing member (male side of the surface fastener) disposed on the outer surface side of the support portion 143 to secure the support portion 143 and the belt portion 141 via the respective securing members 152 and 153.

Subsequently, as illustrated in FIG. 6, the operator, etc. passes the first arm portion 130 between the thumb f1 and the index finger f2 of the hand H of the patient. Then, as illustrated in FIG. 7, the operator, etc. overlaps a part of the first arm portion 130 on the belt portion 141 near the limb of the patient on the palm Hp side of the hand H of the patient. The operator, etc. brings the first securing member (female side of the surface fastener) 151 disposed on the inner surface side of the first arm portion 130 into contact with the second securing member (male side of the surface fastener) disposed on the outer surface side of the belt portion 141 to secure the belt portion 141 and the first arm portion 130 via the respective securing members 151 and 152.

Subsequently, the operator, etc. connects the syringe to the injection portion 191, and injects air into the second inflatable portion 180 to inflate the first inflatable portion 170 and the second inflatable portion 180. In the hemostatic device 100, when the first inflatable portion 170 and the second inflatable portion 180 are inflated, the first inflatable portion 170 applies a compressive force to the puncture site t1. After inflating the respective inflatable portions 170 and 180, as illustrated in FIGS. 8 and 9, the operator, etc. removes the sheath tube of the introducer 200 from the puncture site t1.

The operator, etc. confirms that there is no bleeding from the puncture site t1 while hemostasis is performed using the hemostatic device 100. When there is bleeding from the puncture site t1, the amount of air injected into the respective inflatable portions 170 and 180 is adjusted.

After a certain period of time passes after start of hemostasis, the operator, etc. gradually depressurizes the respective inflatable portions 170 and 180 to confirm that hemostasis at the puncture site t1 is properly performed. After the hemostasis at the puncture site t1 is completed, the operator, etc. sufficiently depressurizes the respective inflatable portions 170 and 180. Then, the operator, etc. releases securing of the hemostatic device 100 by the first arm portion 130 and the second arm portion 140, and removes the hemostatic device 100 from the hand H of the patient.

As illustrated in FIG. 8, during hemostasis, the hemostatic device 100 is firmly secured to the hand H of the patient by the first arm portion 130 passed between the thumb f1 and index finger f2 of the hand H of the patient and the second arm portion 140 wrapped around the limb of the patient. For this reason, since each finger is not covered by the covering member 110 in a state where the hemostatic device 100 is attached to the hand H, the patient can freely move the finger(s) while hemostasis is performed.

The operator, etc. can easily attach the hemostatic device 100 to the hand H of the patient by proceeding with an attachment operation according to the procedure described with reference to FIGS. 4 to 7. Therefore, the operator can easily attach the hemostatic device 100 to the patient in a short time using the first arm portion 130 and the second arm portion 140. Further, the hemostatic device 100 can be secured to the hand H of the patient by securing the arm portions 130 and 140 to each other using the securing members 151, 152 and 153 disposed on the respective arm portions 130 and 140. Therefore, the hemostatic device 100 can prevent damage to the skin of the patient during attachment when compared to a hemostatic device secured to the hand H or the forearm A of the patient using a seal member provided with an adhesive material.

Further, since the hemostatic device 100 includes the first inflatable portion 170 as a member that applies a compressive force to the puncture site t1, the compressive force can be easily adjusted by adjusting the internal pressure of the first inflatable portion 170. Further, in the hemostatic device 100, even when the first inflatable portion 170 is deformed so as to change the internal pressure following movement of the hand H when the patient moves the hand H, the deformable second inflatable portion 180 mitigates the change in the internal pressure of the first inflatable portion 170. Therefore, the first inflatable portion 170 has a high following property (conformability) to movement of the hand H of the patient, and compression on the puncture site t1 by the first inflatable portion 170 can be appropriately maintained.

Further, the hemostatic device 100 is configured to cover only a part of the dorsal side Hb of the hand H of the patient by the covering member 110, and is not configured to cover the entire hand H. For this reason, when the patient moves the hand H in a state where the hemostatic device 100 is attached to the hand H of the patient, it is possible to prevent movement of the hand H of the patient from being transmitted to the entire hemostatic device 100. Therefore, the hemostatic device 100 can suppress position shift from the hand H of the patient when the patient moves the hand H in the state where the hemostatic device 100 is attached to the hand H of the patient.

The hemostatic device 100 can be attached to, for example, the right hand of the patient without changing the configuration of the hemostatic device 100. When the hemostatic device 100 is attached to the right hand of the patient, an attachment procedure is substantially the same as the procedure illustrated in FIGS. 4 to 7.

Hereinafter, effects of the present embodiment will be described.

The hemostatic device 100 according to the present embodiment includes the covering member 110 configured to be disposed to cover the puncture site t1 on the hand H of the patient, the securing members 151, 152, and 153 for securing the covering member 110 in a state where the covering member 110 covers the puncture site t1, and the inflatable member 160 connected to the covering member 110 and configured to be inflated by injection of a fluid. The covering member 110 includes the main body 120 to which the inflatable member 160 is connected, the first arm portion 130 protruding from the main body 120, and the second arm portion 140 protruding from the main body 120 in a direction different from the longitudinal direction of extent of the first arm portion 130. The first arm portion 130 is configured to be secured to the second arm portion 140 by passing between the fingers f1 and f2 of the patient in a state where the second arm portion 140 is wrapped around the limb of the patient, the second arm portion 140 includes the belt portion 141 for wrapping around the limb of the patient and the support portion 143 located on the straight line c1 connecting the first arm portion 130 and the inflatable member 160 to connect the main body 120 and the belt portion 141, and the belt portion 141 is configured to be secured to the support portion 143 in a state of being wrapped around the limb of the patient.

The hemostatic device 100 configured as described above can secure the main body 120, to which the inflatable member 160 is connected, to the puncture site t1 by wrapping the belt portion 141 of the second arm portion 140 around the limb of the patient. Further, in the hemostatic device 100, when the first arm portion 130 is secured to the second arm portion 140 by passing between the fingers f1 and f2 of the patient, it is possible to prevent the inflatable member 160 from rising on the distal side of the hand H while preventing position shift of the inflatable member 160. Further, when the hemostatic device 100 is attached to the hand H of the patient, it is possible to prevent the inflatable member 160 from rising on the proximal side of the hand H by connecting the belt portion 141 of the second arm portion 140 to the support portion 143 located on the straight line c1 connecting the first arm portion 130 and the inflatable member 160. For this reason, in the hemostatic device 100, even when the patient moves the hand H in a state in which the inflatable member 160 is inflated, by securing the first arm portion 130 and the second arm portion 140 and securing the belt portion 141 and the support portion 143, rising of the distal side (fingertip side) and the proximal side (wrist side) of the main body 120 to which the inflatable member 160 is connected is prevented. Thus, it is possible to appropriately maintain the compressive force of the inflatable member 160 on the puncture site t1 formed on the dorsal side Hb of the hand H of the patient.

Further, the main body 120 of the covering member 110 has the first region 121 and the second region 122 which is formed in a part different from the first region 121 and in which the inflatable member 160 is disposed. The second region 122 has the support member 125 having a higher rigidity than that of the first region 121. Therefore, the hemostatic device 100 can prevent the inflatable member 160 from rising from the dorsal side Hb of the hand H of the patient by the support member 125 while hemostasis is performed. As a result, the hemostatic device 100 can suitably apply a compressive force from the inflatable member 160 to the puncture site t1 while being attached to the hand H of the patient.

Further, the first region 121 of the main body 120 is disposed to surround the periphery of the second region 122 of the main body 120. The support member 125 is inserted into the insertion portion 128a disposed on the outer surface side of the second region 122. Since the support member 125 is surrounded by the first region 121 which is more flexible than the support member 125, when the inflatable member 160 located in the second region 122 is inflated, a force expanding from the inside to the outside of the inflatable member 160 is hardly transmitted from the support member 125 to the first region 121. Therefore, the hemostatic device 100 can suppress formation of a gap between the surface of the dorsal side Hb of the hand H and the main body 120 at the peripheral edge of the main body 120. Further, by disposing the support member 125 to cover the outer surface side of the second region 122 of the main body 120, it is possible to effectively prevent the inflatable member 160 from rising from the dorsal side Hb of the hand H of the patient. Further, the hemostatic device 100 can be equipped with the support member 125 by a simple operation of inserting the support member 125 into the insertion portion 128a.

Further, the support member 125 has the curved portion 125a formed on the first arm portion 130 side of the support member 125. For this reason, in the support member 125, when the inflatable member 160 is inflated, a direction in which the inflatable member 160 applies a compressive force to the hand H of the patient is directed in an oblique direction toward the puncture site t1. For this reason, the hemostatic device 100 can more effectively apply a compressive force to the puncture site t1. Further, when the curved portion 125a of the support member 125 wraps the end portion of the inflatable member 160 on the first arm portion 130 side, the inflatable member 160 directs the compressive force toward the center side of the support member 125, and thus the compressive force of the inflatable member 160 is inhibited from escaping to the outside of the support member 125. As a result, even in a case where a gap is generated between the first arm portion 130 and the hand H of the patient, when the curved portion 125a presses the inflatable member 160 against the skin of the hand H of the patient, it is possible to prevent the inflatable member 160 from rising, and it is possible to suppress a decrease in the compressive force of the inflatable member 160 applied to the puncture site t1.

Further, the inflatable member 160 is secured to the covering member 110 on the curved portion 125a side of the support member 125. For this reason, in the inflatable member 160 (the first inflatable portion 170 and the second inflatable portion 180), by securing the inflatable member 160 and the support member 125, position shift of the inflatable member 160 with respect to the curved portion 125a of the support member 125 is prevented, and thus the compressive force of the curved portion 125a on a portion close to the first arm portion 130 becomes stronger. As a result, in the hemostatic device 100, even when a gap is generated between the first arm portion 130 and the surface of the hand H due to bending of the thumb f1 or the wrist of the patient, it is possible to prevent a decrease in the compressive force by the inflatable member 160.

Further, the inflatable member 160 has the first inflatable portion 170 and the deformable auxiliary member 180 having a smaller outer shape than that of the first inflatable portion 170, and the auxiliary member 180 is disposed to overlap the first inflatable portion 170 on the first arm portion 130 side of the first inflatable portion 170. When the first inflatable portion 170 inflates, the second inflatable portion 180 inhibits the first inflatable portion 170 from rising from the body surface of the patient. Therefore, when the first inflatable portion 170 inflates, the first inflatable portion 170 can effectively apply the compressive force to the puncture site t1. Further, when the patient bends a finger (for example, the thumb f1) or the wrist downward (to the palm Hp side of the hand H), the hemostatic device 100 maintains a state in which the auxiliary member 180 is deformed by following movement of the finger or the wrist and the auxiliary member 180 applies the compressive force to the puncture site t1. Further, in the hemostatic device 100, when the patient bends a finger (for example, the thumb f1) or the wrist upward (to the side of the dorsal side Hb of the hand H), the auxiliary member 180 is deformed following movement of the finger or the wrist, and the compressive force applied from the auxiliary member 180 to the puncture site t1 is weakened. Therefore, while hemostasis is performed by the hemostatic device 100, the hemostatic device 100 can maintain a state in which an appropriate compressive force is applied to the puncture site t1 while allowing the patient to move the finger or the wrist relatively freely.

Further, the auxiliary member 180 is the second inflatable portion 180 configured to be inflated by injection of a fluid, and the lumen 181 of the second inflatable portion 180 communicates with the lumen 171 of the first inflatable portion 170. Since the auxiliary member 180 includes the second inflatable portion 180 which is inflatable, it is possible to improve the following property of the second inflatable portion 180 to movement of the hand H. Further, since the lumen 171 of the first inflatable portion 170 and the lumen 181 of the second inflatable portion 180 communicate with each other, the first inflatable portion 170 and the second inflatable portion 180 can be easily inflated.

Further, the hemostatic device 100 has the injection portion 191 for injecting a fluid into the inflatable member 160. The cushioning member 192 having an inflatable space is disposed between the injection portion 191 and the inflatable member 160. In the hemostatic device 100, when the patient moves the hand H while the hemostatic device 100 is attached to the patient, the inflatable member 160 (the first inflatable portion 170 and the second inflatable portion 180) compressing the puncture site t1 on the hand H is deformed. When there is no escape place for air in the lumen of the inflatable member 160 (the lumen 171 of the first inflatable portion 170 and the lumen 181 of the second inflatable portion 180) during deformation of the inflatable member 160, deformation of the inflatable member 160 is hindered. Therefore, the patient has a limited movable range of the hand H. The cushioning member 192 included in the hemostatic device 100 allows air to move from the lumen of the inflatable member 160 to the cushioning member 192 when the patient moves the hand H. Therefore, it is possible to prevent the movable range from being restricted by the inflatable member 160 when the patient moves the hand H. When the patient returns the hand H from the deformed state to the original state, air moves from the cushioning member 192 to the inflatable member 160. Therefore, the compressive force can be effectively applied to the puncture site t1 from the inflatable member 160.

(Modification)

Next, a description will be set forth of a modification of the hemostatic device according to the first embodiment described above. In description of the modification, features that are the same or similar to those described above are identified by the same reference numerals and a detailed description of such features is not repeated. Thus, content not particularly described in the description of the modification can be regarded as the same as that in the first embodiment.

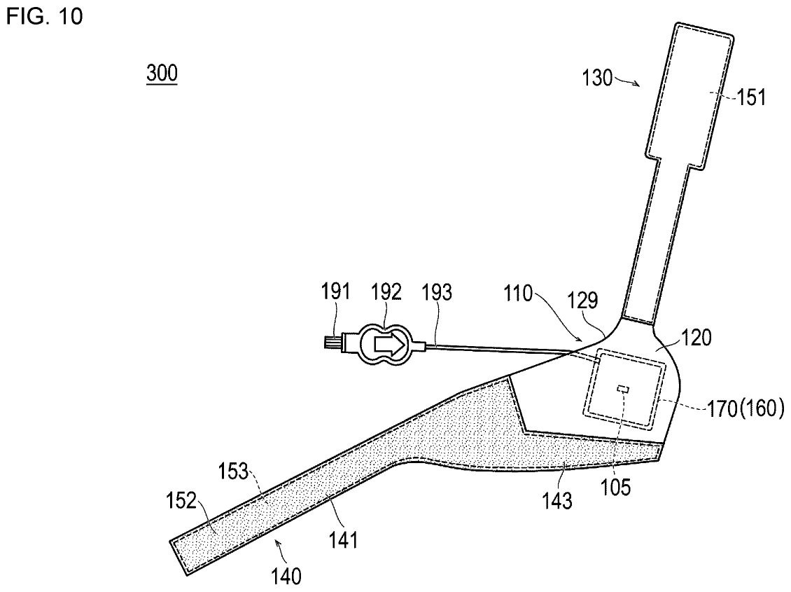

FIG. 10 illustrates a hemostatic device 300 according to the modification. In the hemostatic device 300 according to the modification, an inflatable member 160 includes only a first inflatable portion 170. That is, the inflatable member 160 does not include the second inflatable portion 180. Similarly to the hemostatic device 100 described above, by securing the hemostatic device 300 configured in this way to the hand H of the patient using respective arm portions 130 and 140 and a support portion 143, it is possible to prevent the inflatable member 160 from rising from the hand H during hemostasis. As a result, in the hemostatic device 300, the inflatable member 160 can effectively apply a compressive force to the puncture site t1.

In the hemostatic device 300 according to the modification, the first inflatable portion 170 is directly fixed to an inner surface of a covering member 110. Further, a tube 193 is connected so as to communicate with a lumen 171 of the first inflatable portion 170. Other configurations, features, or aspects of the hemostatic device 300 are substantially the same as those of the hemostatic device 100 described above.

### Second Embodiment

Next, a description will be given of a hemostatic device according to a second embodiment. In description of the second embodiment, features that are the same or similar to those described above are identified by the same reference numerals and a detailed description of such features is not repeated. In addition, content not particularly described in the description of the second embodiment can be regarded as the same as that in the first embodiment.

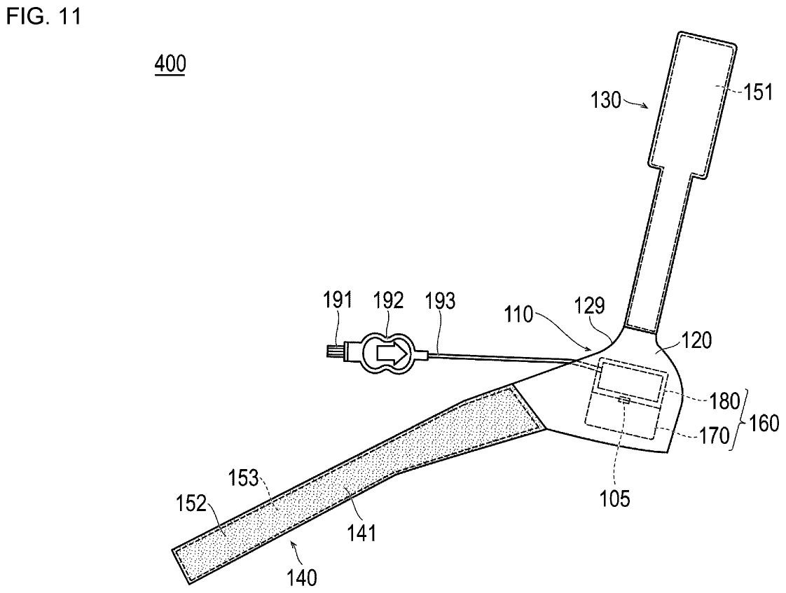

FIG. 11 illustrates a hemostatic device 400 according to the second embodiment. In the hemostatic device 400 according to the second embodiment, a second arm portion 140 does not include the support portion 143. In this respect, the hemostatic device 400 differs from the hemostatic device 100 according to the first embodiment.

As the time of attaching the hemostatic device 400 to the patient, the operator wraps a belt portion 141 of the second arm portion 140 around the limb of the patient. In this instance, the operator brings a third securing portion (female side of a surface fastener) 153 disposed on an inner surface of the belt portion 141 into contact with a second securing portion (male side of the surface fastener) 152 disposed on an outer surface of the belt portion 141, thereby securing the respective securing members 152 and 153. The operator, etc. holds the belt portion 141 in a state of being wrapped around the limb of the patient by securing the respective securing members 152 and 153. Subsequently, the operator can attached the hemostatic device 400 to the hand H of the patient by securing the first arm portion 130 to the second arm portion 140 according to a procedure similar to the procedure described in the above embodiment and shown in FIGS. 6 and 7.

The hemostatic device 400 according to the second embodiment includes a covering member 110 configured to be disposed to cover the puncture site t1 on the hand H of the patient, securing members 151, 152, and 153 for securing the covering member 110 in a state where the covering member 110 covers the puncture site t1, and an inflatable member 160 connected to the covering member 110 and configured to be inflated by injection of a fluid. The covering member 110 includes a main body 120 to which the inflatable member 160 is connected, a first arm portion 130 protruding or extending from the main body 120, and a second arm portion 140 protruding or extending from the main body 120 in a direction different from a longitudinal direction of extent of the first arm portion 130. The first arm portion 130 is configured to be secured to the second arm portion 140 by passing between the fingers f1 and f2 of the patient in a state where the second arm portion 140 is wrapped around the limb of the patient. The inflatable member 160 has the first inflatable portion 170 and the deformable auxiliary member 180 having a smaller outer shape than that of the first inflatable portion 170, and the auxiliary member 180 is disposed to overlap the first inflatable portion 170 on the first arm portion 130 side of the first inflatable portion 170.

In the hemostatic device 400 according to the second embodiment, the main body 120 to which the first inflatable portion 170 is connected can be secured to the puncture site t1 by wrapping the second arm portion 140 around the limb of the patient. Further, the hemostatic device 400 can prevent the first inflatable portion 170 from rising on the distal side of the hand H while preventing the first inflatable portion 170 from being shifted by securing the first arm portion 130 to the second arm portion 140 through between the fingers f1 and f2 of the patient. Further, when the first inflatable portion 170 inflates, the hemostatic device 400 inhibits the first inflatable portion 170 from rising in a direction away from the puncture site t1 on the hand H of the patient by the auxiliary member 180. As a result, the hemostatic device 400 can appropriately secure the first inflatable portion 170 and the auxiliary member 180 to the puncture site t1. Further, in the hemostatic device 400, when the first inflatable portion 170 inflates, the auxiliary member 180 inhibits the first inflatable portion 170 from rising in the direction away from the puncture site t1 of the hand H of the patient, and thus it is unnecessary to secure to firmly tighten the first arm portion 130 passing between the fingers f1 and f2 of the patient to the second arm portion 140. In this way, the patient can move the finger or the wrist relatively freely in a state where the hemostatic device 400 is attached.

Further, when the patient bends a finger (for example, the thumb f1) or the wrist downward (the palm Hp side of the hand H), the hemostatic device 400 maintains a state in which the auxiliary member 180 is deformed by following movement of the finger or the wrist and the auxiliary member 180 applies the compressive force to the puncture site t1. Further, in the hemostatic device 400, when the patient bends a finger (for example, the thumb f1) or the wrist upward (to the side of the dorsal side Hb of the hand H), the auxiliary member 180 is deformed following movement of the finger or the wrist, and the compressive force applied from the auxiliary member 180 to the puncture site t1 is weakened. Therefore, while hemostasis is performed by the hemostatic device 400, the hemostatic device 400 can maintain a state in which an appropriate compressive force is applied to the puncture site t1 while allowing the patient to move the finger or the wrist relatively freely.

Further, the main body 120 of the covering member 110 has the first region 121 and the second region 122 which is formed in a part different from the first region 121 and in which the inflatable member 160 is disposed. The second region 122 has the support member 125 having a higher rigidity than that of the first region 121. Therefore, the hemostatic device 400 can prevent the inflatable member 160 from rising from the dorsal side Hb of the hand H of the patient by the support member 125 while hemostasis is performed. As a result, in the hemostatic device 400, the inflatable member 160 can suitably apply a compressive force to the puncture site t1 in a state of being attached to the hand H of the patient.

Further, the first region 121 of the main body 120 is disposed to surround the periphery of the second region 122 of the main body 120. The support member 125 is inserted into the insertion portion 128a disposed on the outer surface side of the second region 122. Since the support member 125 is surrounded by the first region 121 which is more flexible than the support member 125, when the inflatable member 160 located in the second region 122 is inflated, a force expanding from the inside to the outside of the inflatable member 160 is hardly transmitted from the support member 125 to the first region 121. Therefore, the hemostatic device 400 can suppress formation of a gap between the surface of the hand H of the patient and the main body 120 at the peripheral edge of the main body 120. Further, by disposing the support member 125 to cover the outer surface side of the second region 122 of the main body 120, it is possible to effectively prevent the inflatable member 160 from rising from the dorsal side Hb of the hand H of the patient. Further, the hemostatic device 400 can be equipped with the support member 125 by a simple operation of inserting the support member 125 into the insertion portion 128a.

Further, the support member 125 has the curved portion 125a formed on the first arm portion 130 side of the support member 125. For this reason, in the support member 125, when the inflatable member 160 is inflated, a direction in which the inflatable member 160 applies a compressive force to the hand H of the patient is directed in an oblique direction toward the puncture site t1. For this reason, the hemostatic device 100 can more effectively apply a compressive force to the puncture site t1. Further, when the curved portion 125a of the support member 125 wraps the end portion of the inflatable member 160 on the first arm portion 130 side, the inflatable member 160 directs the compressive force toward the center side of the support member 125, and thus the compressive force of the second inflatable portion 180 is inhibited from escaping to the outside of the support member 125. As a result, even in a case where a gap is generated between the first arm portion 130 and the hand H of the patient, when the curved portion 125a presses the second inflatable portion 180 against the skin, it is possible to prevent the first inflatable portion 170 from rising, and it is possible to suppress a decrease in the compressive force of the inflatable member 160 applied to the puncture site t1.

Further, the inflatable member 160 is fixed to the covering member 110 on the first arm portion 130 side. Specifically, the second inflatable portion 180 of the inflatable member 160 is fixed to the covering member 110 on the first arm portion 130 side. For this reason, the inflatable member 160 (second inflatable portion 180) is fixed to the covering member 110 on the first arm portion 130 side, so that when the inflatable member 160 inflates, the direction in which the inflatable member 160 applies the compressive force to the hand H of the patient is directed in an oblique direction toward the puncture site t1. Therefore, the hemostatic device 100 can more effectively apply the compressive force to the puncture site t1.

Further, the inflatable member 160 is fixed to the covering member 110 on the curved portion 125a side of the support member 125. For this reason, in the inflatable member 160 (the first inflatable portion 170 and the second inflatable portion 180), position shift with respect to the curved portion 125a of the support member 125 of the inflatable member 160 is prevented by securing the inflatable member 160 and the support member 125, and thus a compressive force of the curved portion 125a on a position close to the first arm portion 130 becomes stronger. In this way, even when a gap is created between the surface of the first arm portion 130 and the surface of the hand H due to bending of the thumb f1 or the wrist of the patient, the hemostatic device 100 can prevent a decrease in the compressive force by the inflatable member 160.

Further, the auxiliary member 180 is a second inflatable portion 180 configured to be inflated by injection a fluid, and the lumen 181 of the second inflatable portion 180 communicates with the lumen 171 of the first inflatable portion 170. Since the auxiliary member 180 includes the second inflatable portion 180 which is inflatable, it is possible to improve the following property of the second inflatable portion 180 to movement of the hand H. Further, since the lumen 171 of the first inflatable portion 170 and the lumen 181 of the second inflatable portion 180 communicate with each other, the first inflatable portion 170 and the second inflatable portion 180 can be easily inflated.

Further, the first inflatable portion 170 and the second inflatable portion 180 have communication holes 172 and 182 communicating with the lumen 171 of the first inflatable portion 170 and the lumen 181 of the second inflatable portion 180, respectively. In addition, in the first inflatable portion 170, only a certain range around each of the communication holes 172 and 182 is fixed to the second inflatable portion 180. Specifically, in the first inflatable portion 170, only a certain range around each of the communication holes 172 and 182 located near the central portion of the second inflatable portion 180 is fixed to the second inflatable portion 180. Further, the first inflatable portion 170 is not directly connected to the covering member 110, and is indirectly connected to the covering member 110 via the second inflatable portion 180. In this way, in the second inflatable portion 180, the peripheral edge of the second inflatable portion 180 is not fixed to the first inflatable portion 170, and thus the outer shape of the second inflatable portion is freely deformed. Therefore, even when the patient bends a finger (for example, the thumb f1) or the wrist upward (to the side of the dorsal side Hb of the hand H), the hemostatic device 400 can maintain a large area in which the second inflatable portion 180 compresses the first inflatable portion 170. Therefore, the hemostatic device 400 can effectively apply a compressive force to the puncture site t1 from the inflatable member 160.

Further, the hemostatic device 400 has an injection portion 191 for injecting a fluid into the inflatable member 160. A cushioning member 192 having an inflatable space is disposed between the injection portion 191 and the inflatable member 160. In the hemostatic device 400, when the patient moves the hand H while the hemostatic device 400 is attached to the patient, the inflatable member 160 (the first inflatable portion 170 and the second inflatable portion 180) that compresses the puncture site t1 of the hand H is deformed. When there is no escape place for air in the lumen of the inflatable member 160 (the lumen 171 of the first inflatable portion 170 and the lumen 181 of the second inflatable portion 180) during deformation of the inflatable member 160, deformation of the inflatable member 160 is hindered. Therefore, the patient has a limited movable range of the hand H. The cushioning member 192 included in the hemostatic device 400 allows air to move from the lumen of the inflatable member 160 to the cushioning member 192 when the patient moves the hand H. Therefore, it is possible to prevent the movable range from being restricted by the inflatable member 160 when the patient moves the hand H. When the patient returns the hand H from the deformed state to the original state, air moves from the cushioning member 192 to the inflatable member 160. Therefore, the compressive force can be effectively applied to the puncture site t1 from the inflatable member 160.

(First Modification)

Next, a description will be set forth of modifications of the hemostatic device according to the second embodiment described above. In the following description of the modifications, features that are the same or similar to those described above are identified by the same reference numerals and a detailed description of such features is not repeated. In addition, content not particularly described in the description of the modifications can be regarded as the same as that in the embodiments described above.

FIG. 12 illustrates a hemostatic device 500 according to a first modification. The hemostatic device 500 according to the first modification has a securing auxiliary portion 529 disposed in the main body 120 of the covering member 110. The securing auxiliary portion 529 is disposed at a position different from a position where the first arm portion 130 is disposed and different from a position where the second arm portion 140 is disposed in the main body 120. Specifically, the securing auxiliary portion 529 is disposed at a position facing the second arm portion 140 with the main body 120 interposed therebetween.

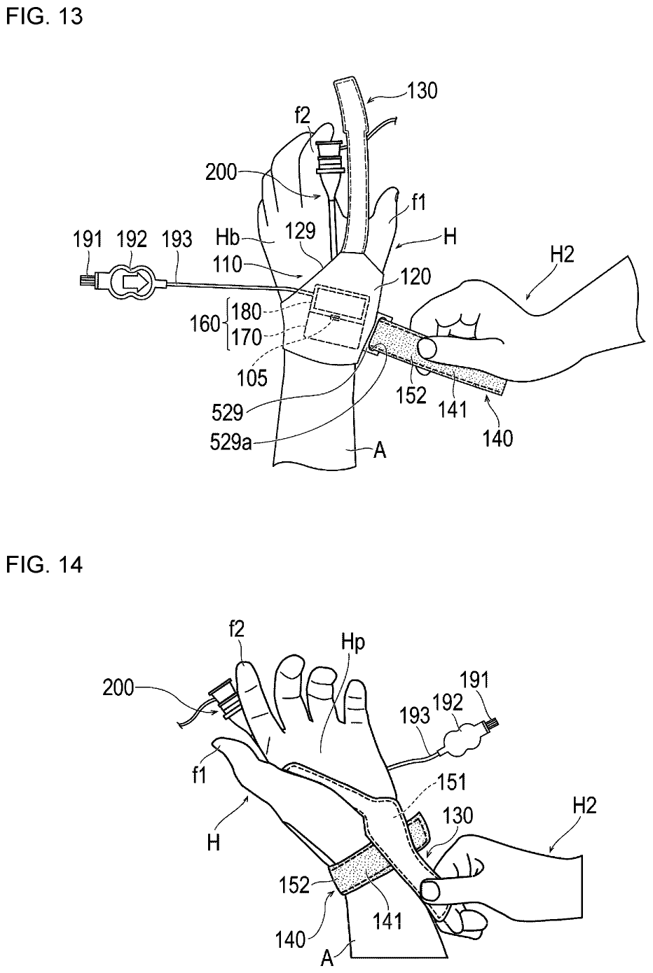

The securing auxiliary portion 529 is made of a material more rigid than that of the main body 120. A hole 529a through which the belt portion 141 of the second arm portion 140 can be inserted is formed in the securing auxiliary portion 529. As illustrated in FIG. 12, the hole 529a is tilted at an angle along a side portion of the main body 120 (tilted from a diagonally upper left side to a diagonally lower right side in the figure) in a state of not being attached to the hand H of the patient. Since the hole 529a is tilted in this way, as illustrated in FIG. 13, the operator, etc. can easily insert the belt portion 141 of the second arm portion 140 into the hole 529a at the time of attaching the hemostatic device 500 to the patient.

The securing auxiliary portion 529 may have a hook structure that includes a hole and a slit communicating with the hole instead of the hole 529a. Further, the securing auxiliary portion 529 may have a hole formed in a flexible material.

For example, the hole 529a can be disposed so that one end portion of the hole 529a is located on the proximal side (right side in FIG. 12) of the curved portion 125a of the support member 125. Further, the hole 529a can be disposed so as to be substantially parallel to the first inflatable portion 170. By disposing the hole 529a in this way, a tightening force is easily transmitted to the first inflatable portion 170 when the belt portion 141 of the second arm portion 140 is wrapped around the limb of the patient.

For example, the securing auxiliary portion 529 can be connected to the main body 120 by adhesion or welding.

As illustrated in FIG. 13, at the time of attaching the hemostatic device 500 to the hand H of the patient, the operator, etc. inserts the belt portion 141 of the second arm portion 140 into the hole 529a of the securing auxiliary portion 529. The operator, etc. folds back the belt portion 141 through which the hole 529a of the securing auxiliary portion 529 is inserted toward the palm Hp side of the hand H of the patient. In a state where a part of the belt portion 141 is folded back, the operator, etc. brings the third securing portion (female side of the surface fastener) 153 disposed on the inner surface of the belt portion 141 into contact with the second securing portion (male side of the surface fastener) 152 disposed on the outer surface of the belt portion 141, thereby securing the respective securing members 152 and 153. The operator, etc. holds the belt portion 141 in a state of being wrapped around the limb of the patient by securing the respective securing members 152 and 153. As illustrated in FIG. 14, the operator, etc. can attach the hemostatic device 500 to the hand H of the patient by securing the first arm portion 130 passing between the respective fingers f1 and f2 to the second arm portion 140.

(Second Modification)

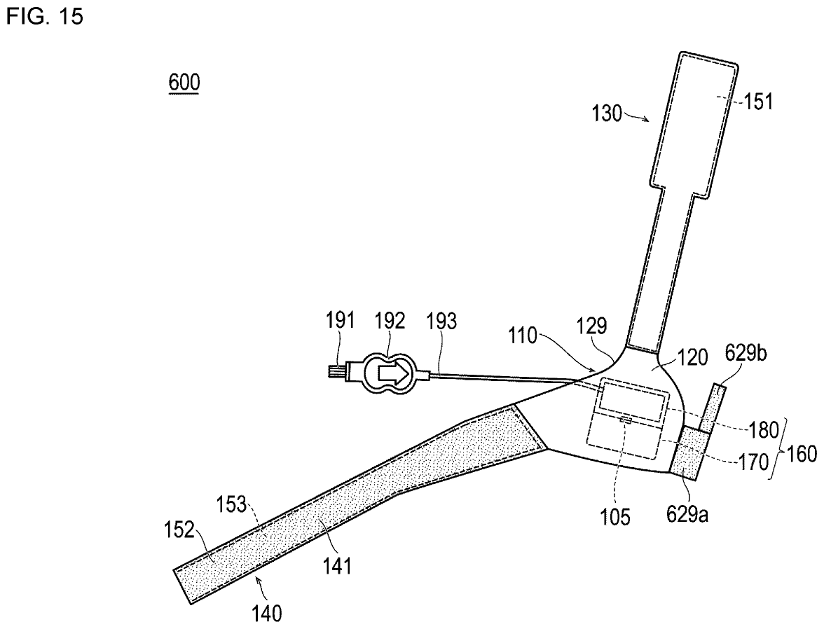

FIG. 15 illustrates a hemostatic device 600 according to a second embodiment. The hemostatic device 600 according to the second modification is different from the hemostatic device 500 according to the first modification described above in a configuration of securing auxiliary portions 629a and 629b.

The first securing auxiliary portion 629a is connected to the main body 120. A male side of a surface fastener is disposed on an outer surface (outer surface on a plan view illustrated in FIG. 15) of the first securing auxiliary portion 629a.

The second securing auxiliary portion 629b is connected to the first securing auxiliary portion 629a. A female side of the surface fastener is disposed on an outer surface (outer surface on the plan view illustrated in FIG. 15) of the second securing auxiliary portion 629b.

The respective securing auxiliary portions 629a and 629b can be connected to the main body 120 by, for example, adhesion or welding. Installation of the second securing auxiliary portion 629b may be omitted as appropriate.

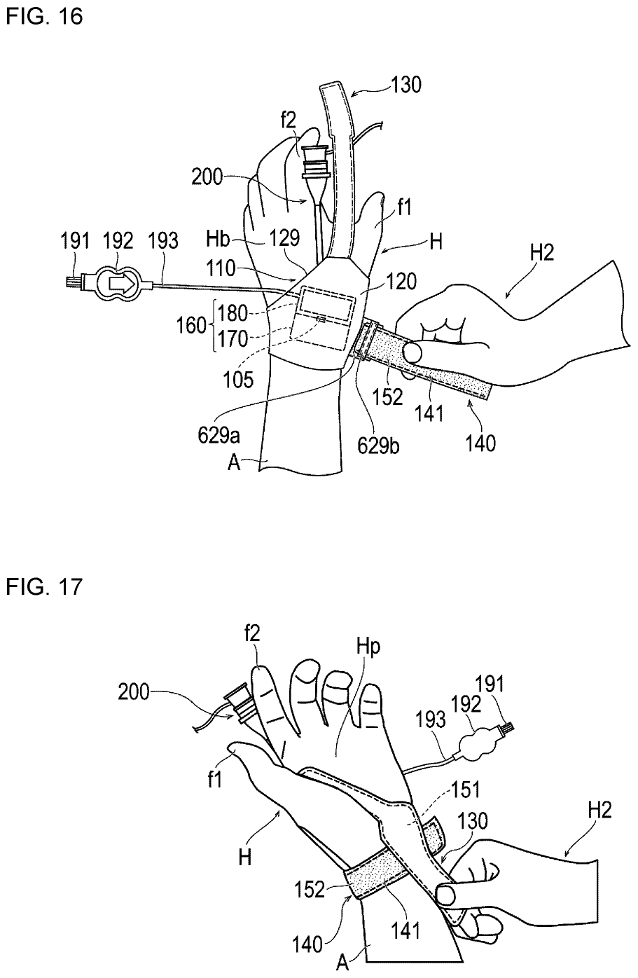

As illustrated in FIG. 16, at the time of attaching the hemostatic device 600 to the hand H of the patient, the operator, etc. secures the third securing member (female side of the surface fastener) 153 disposed on the inner surface of the belt portion 141 of the second arm portion 140 to the first securing auxiliary portion 629a. Subsequently, the operator, etc. folds back the belt portion 141 toward the palm Hp side of the hand H of the patient using a portion of the belt portion 141 secured to the first securing auxiliary portion 629a as a base point.

The operator, etc. disposes the female side of the surface fastener disposed on the outer surface of the second securing auxiliary portion 629b to cover a part of the belt portion 141 secured to the first securing auxiliary portion 629a, and secures the second securing auxiliary portion 629b to the belt portion 141.

Subsequently, the operator, etc. wraps a folded part of the belt portion 141 around the limb toward the palm Hp side. In this instance, the operator, etc. brings the third securing portion (female side of the surface fastener) 153 disposed on the inner surface of the belt portion 141 into contact with the second securing portion (male side of the surface fastener) 152 disposed on the outer surface of the belt portion 141, thereby securing the respective securing members 152 and 153. The operator, etc. holds the belt portion 141 in a state of being wrapped around the limb of the patient by securing the respective securing members 152 and 153.

As illustrated in FIG. 17, the operator, etc. can attach the hemostatic device 600 to the hand H of the patient by securing the first arm portion 130 passing between the respective fingers f1 and f2 to the second arm portion 140.

Even though the hemostatic device disclosed here has been described above through the embodiments, the invention is not limited to content described in this specification, and can be appropriately modified based on description of the scope of claims.

In the description of the embodiments, the hemostatic device for performing hemostasis at the puncture site formed on the dorsal side of the left hand has been exemplified. However, the hemostatic device can be used to perform hemostasis on a puncture site formed on a dorsal side of a right hand, a puncture site formed on a palm of the right hand, a puncture site formed on a palm of the left hand, etc.

Further, the auxiliary member is not limited to the inflatable member described in each embodiment. For example, the auxiliary member may include a member made of a resin material such as plastic, gel, etc., a member containing gel whose moisture content decreases over time to gradually reduce a compressive force, an elastic material such as a sponge-like substance, an aggregate of fibers such as cotton, metal, a member having a predetermined three-dimensional shape (sphere, ellipsoid, triangular pyramid, etc.), an appropriate combination thereof, etc.

In addition, the shape and dimensions of each portion of the hemostatic device are not particularly limited as long as the inflatable member can be disposed at the puncture site, the first arm portion can be disposed between arbitrary fingers, and the second arm portion can be disposed on the limb, and changes can be made as appropriate.

The detailed description above describes embodiments of a hemostatic device and method of use representing examples of the inventive hemostatic device and method disclosed here. The invention is not limited, however, to the precise embodiments, modifications and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A hemostatic device comprising:
a covering member configured to cover a site on a hand of a patient where bleeding is to be stopped;
a securing member connected to the covering member and configured to secure the covering member on the hand of the patient while the covering member is covering the site on the patient's hand where bleeding is to be stopped;
an inflatable member connected to the covering member and configured to be inflated by injection of a fluid;
the covering member including a main body to which the inflatable member is connected, a first arm portion extending away from the main body in a longitudinal direction of extent, and a second arm portion extending away from the main body in a direction different from the longitudinal direction of extent of the first arm portion;
the first arm portion and the inflatable member being positioned so that a straight line connects the first arm portion and the inflatable member;
the second arm portion includes a belt portion configured to be wrapped around a limb of the patient and a support portion that connects the main body and the belt portion, the support portion being located on the straight line that connects the first arm portion and the inflatable member;
the first arm portion being configured to be secured to the second arm portion by passing between fingers of the patient's hand in a state in which the second arm portion is wrapped around the limb of the patient; and
the belt portion of the second arm portion being configured to be secured to the support portion in the state in which the second arm portion is wrapped around the limb of the patient.

2. The hemostatic device according to claim 1,
wherein the main body includes a first region and a second region, the second region being located in a part of the main body different from the first region, the inflatable member being disposed in the second region, and
the second region including a support member having a higher rigidity than a rigidity of the first region.

3. The hemostatic device according to claim 2,
further comprising a cover member connected to the covering member so that a space exists between the cover and the main body of the covering member, the cover being located on one side of the main body of the covering member and the inflatable member being located on an opposite side of the main body of the covering member;
wherein the first region surrounds a periphery of the second region, and
the support member is located in the space.

4. The hemostatic device according to claim 2, wherein the support member possesses a longitudinal extent and includes one end portion closer to the first arm portion and an opposite end located farther from the first arm portion, the support member including a curved portion located at the one end portion of the support member.

5. The hemostatic device according to claim 4, wherein the inflatable member is fixed to the covering member so that the curved portion of the support member overlies the inflatable member.

6. The hemostatic device according to claim 1,
wherein the inflatable member includes a first inflatable portion and a deformable auxiliary member having a smaller outer shape than an outer shape of the first inflatable portion,
the first inflatable member includes one portion located closer to the first arm portion than an other portion of the first inflatable member, and
the auxiliary member overlaps the one portion of the first inflatable portion that is located closer to the first arm portion.

7. The hemostatic device according to claim 6,
wherein the auxiliary member is a second inflatable portion configured to be inflated by injection of a fluid,
the first inflatable portion includes a lumen, and the second inflatable portion includes a lumen, and
the lumen of the second inflatable portion communicates with the lumen of the first inflatable portion so that fluid in the first inflatable portion flows into the second inflatable portion.

8. The hemostatic device according to claim 1, further comprising
an injection portion for injecting a fluid into the inflatable member, and
a cushioning member disposed between the injection portion and the inflatable member, the cushioning member including an inflatable space that is inflatable.

9. The hemostatic device according to claim 1, wherein the main body includes a first region surrounding a second region, and further comprising a support member positioned in a space in the second region of the main body so that the support member is surrounded by the first region of the main body, the support member being more rigid than the first region of the main body,
the support member possessing a longitudinal extent and including one end portion at one end of the longitudinal extent closer to the first arm portion and an opposite end portion located at an opposite end of the support member, the one end portion of the support member located closer to the first arm portion being a curved portion that possesses a concave surface facing the inflatable member and a convex surface facing away from the inflatable member.

10. A hemostatic device comprising:
a covering member configured to cover a site on a hand of a patient where bleeding is to be stopped;
a securing member connected to the covering member and configured to secure the covering member on the hand of the patient while the covering member is covering the site on the patient's hand where bleeding is to be stopped;
an inflatable member connected to the covering member and configured to be inflated by injection of a fluid,
the covering member including a main body to which the inflatable member is connected, a first arm portion extending away from the main body in a longitudinal direction of extent, and a second arm portion extending away from the main body in a direction different from the longitudinal direction of extent of the first arm portion,
the first arm portion is configured to be secured to the second arm portion by passing between fingers of the patient's hand in a state in which the second arm portion is wrapped around a limb of the patient;
the inflatable member including a first inflatable portion and a deformable auxiliary member, the deformable auxiliary member and the deformable auxiliary member each having an outer shape, the deformable auxiliary member having a smaller outer shape than the outer shape of the first inflatable portion;
the first inflatable portion including one portion located closer to the first arm portion and an other portion located farther from the first arm portion; and
the auxiliary member overlapping the one portion of the first inflatable portion that is located closer to the first arm portion.

11. The hemostatic device according to claim 10,
wherein the second arm portion includes a belt portion configured to be wrapped around the limb of the patient, and
the main body includes a securing auxiliary portion disposed at a position different from the first arm portion and the second arm portion, the securing auxiliary portion being configured to be engaged by the belt portion to assist in securing the belt portion wrapped around the limb.

12. The hemostatic device according to claim 10,
wherein the main body includes a first region and a second region, the second region being located a part of the main body different from the first region, the inflatable member being disposed in the second region, and
the second region including a support member having a higher rigidity than a rigidity of the first region.

13. The hemostatic device according to claim 12,
further comprising a cover member connected to the covering member so that a space exists between the cover and the main body of the covering member, the cover being located on one side of the main body of the covering member and the inflatable member being located on an opposite side of the main body of the covering member;
wherein the first region surrounds a periphery of the second region, and
the support member is located in the space.

14. The hemostatic device according to claim 12, wherein the support member possesses a longitudinal extent and includes one end portion closer to the first arm portion and an opposite end located farther from the first arm portion, the support member including a curved portion located at the one end portion of the support member.

15. The hemostatic device according to claim 14, wherein the inflatable member is fixed to the covering member so that the curved portion of the support member overlies the inflatable member.

16. The hemostatic device according to claim 10,
wherein the auxiliary member is a second inflatable portion configured to inflated by injection of a fluid,
the first inflatable portion includes a lumen, and the second inflatable portion includes a lumen, and
the lumen of the second inflatable portion communicates with the lumen of the first inflatable portion so that fluid in the first inflatable portion flows into the second inflatable portion.

17. The hemostatic device according to claim 10, further comprising
an injection portion for injecting a fluid into the inflatable member, and
a cushioning member disposed between the injection portion and the inflatable member, the cushioning member including an inflatable space that is inflatable.

18. The hemostatic device according to claim 10, wherein the main body includes a first region surrounding a second region, and further comprising a support member positioned in a space in the second region of the main body so that the support member is surrounded by the first region of the main body, the support member being more rigid than the first region of the main body,
the support member possessing a longitudinal extent and including one end portion at one end of the longitudinal extent closer to the first arm portion and an opposite end portion located at an opposite end of the support member, the one end portion of the support member located closer to the first arm portion being a curved portion that possesses a concave surface facing the inflatable member and a convex surface facing away from the inflatable member.

19. A method of producing hemostasis at a puncture site on a hand of a patient where bleeding is to be stopped, the method comprising:
positioning a main body of a covering member in overlying relation to the puncture site on the hand of the patient where bleeding is to be stopped so that an inflation member underlying the main body portion is positioned relative to the puncture site, the covering member also including a first arm portion extending away from the main body in a longitudinal direction of extent, and a second arm portion extending away from the main body in a direction different from the longitudinal direction of extent of the first arm portion, the second arm portion including a belt portion and a support portion that connects the belt portion to the main body, the support portion being located on a straight line that connects the first arm portion and the inflatable member;
wrapping the band portion around a limb of the patient and securing the band portion to the support portion to maintain the second arm portion in a wrapped state on the limb of the patient;
passing the first arm portion between two fingers of the hand of the patient while the second arm portion is in the wrapped state on the limb of the patient, and securing a part of the first arm portion which has passed between the two fingers of the hand of the patient to the second arm portion; and
thereafter introducing a fluid into the inflatable member to inflate the inflatable member and apply a compressive force from the inflatable member to the puncture site to perform hemostasis.

20. The method according to claim 19, further comprising a support member positioned in a space located on a side of the main body opposite the inflatable member, the main body including a region positioned outwardly of and completely surrounding the support member, the support member being more rigid than the region of the main body surrounding the support member, the support member possessing a longitudinal extent and including one end portion at one end of the longitudinal extent closer to the first arm portion and an opposite end portion located at an opposite end of the support member, the one end portion of the support member located closer to the first arm portion being a curved portion that possesses a concave surface facing the inflatable member and a convex surface facing away from the inflatable member; and wherein when the inflatable member is inflated the curved portion of the support member directs the compressive force toward a center side of the support member.
